(19) United States
(12) Patent Application Publication
(10) Pub. No.: US 2003/0171820 A1
(43) Pub. Date: Sep. 11, 2003

(54) Bone-implant prosthesis

(76) Inventors: Peter Richard Wilshaw, Oxford (GB); Anna Eva Maria Palsgard, Uppsala (SE)

Correspondence Address: NIXON & VANDERHYE, PC, 1100 N GLEBE ROAD, 8TH FLOOR, ARLINGTON, VA, 22201-4714, US

(21) Appl. No.: 10/332,398

(22) PCT Filed: Jul. 12, 2001
(86) PCT No.: PCT/GB01/03189

(30) Foreign Application Priority Data
Jul. 12, 2000 (GB) 0017148.8

(51) Int. Cl. A61F 2/36; A61F 2/28

(52) U.S. Cl. 623/23.12; 623/23.55; 623/23.57; 623/901

(57) ABSTRACT

A prosthesis is disclosed at least of the surface of which is of metal, said metal being covered by a layer of aluminium oxide which comprises phosphate and/or pores containing bioactive material, optionally with a layer of aluminium or an alloy thereof between the metal and the porous aluminium oxide layer.

The present invention relates to prostheses.

Present prostheses (body implants) for hard tissues e.g. bone and teeth are mainly based on metal implants inserted into bone. These provide excellent mechanical strength but suffer from several general problems.

The implant materials presently used are biologically compatible but biologically inert. This may lead to weak interface with the bone which may even result in the implant working loose—aseptic loosening. Even if this does not occur the interface is so weak it will sometimes not transfer significant tensile stresses to the bone/implant interface and only limited shear stresses. This means that the stress distribution in the natural bone surrounding the implant does not undergo the range of values required to stimulate new bone growth and with time the bone material immediately adjacent to the prosthesis or further away will gradually be absorbed into the body.

At present work is being undertaken to overcome these problems, for example by texturing the surface of the metal implant to provide surface features for the bone to key into. However this solution is not ideal because at the microscopic scale the interface with the bone will be weak since metal is not bioactive. Another approach is to cover the surface of the prosthesis with a bioactive material such as HA (hydroxy apatite), by, for example, spray coating. Problems with this approach include the difficulty in obtaining an HA layer of correct stoichiometry and crystallinity, the weakness of the interface between the implant metal and the HA and the inherent brittleness of the artificial HA itself. Using collagen as a bioactive surface layer is also being tried.

There are materials which are known to provide surfaces that actively promote bone growth; such materials are examples of a class of materials termed “bio-active materials”. The resulting interface with the newly formed bone can be as strong as natural bone itself. Examples of such materials are bioactive glasses and artificial HA. However as yet none of these materials have sufficiently good mechanical properties for them to be used directly as the implant material itself.

According to the present invention there is provided a prosthesis at least a part of the surface of which is of metal, said metal being covered by a layer of aluminium oxide, optionally with a layer of aluminium or an alloy thereof between the metal and the porous aluminum oxide layer, and the aluminium oxide layer either comprises phosphate and/or pores containing bioactive material

Our approach is to cover the implant of metal or other material with a layer of, generally porous, ceramic material which provides an improved substrate for bone growth and attachment. The resulting interface may be of sufficient mechanical strength that the geometry of implants, for example in the case of hip replacements, can be radically altered.

The substrate for the implant is generally of metal typically stainless steel, Co—Cr alloys, titanium or a titanium alloy but other metals which combine the necessary physical properties without any adverse biological effects can be used including aluminium. Specific metals which can be used include stainless steel ASTM No. F745, F55, F56, F138, F139, Co—Cr alloys ASTM No. F75 and F99 which contain molybdenum, F90, which contains tungsten and nickel, and F562, which contains nickel, molybdenum and titanium, and titanium and titanium alloys ASTM No. F67 and F136 which contains aluminium and vanadium.

The total thickness of the aluminium layer is typically from 0.1 to 1000 microns and generally 0.5 or 1 to 10, 20 or 400 microns. The thickness of the porous aluminium oxide layer is typically from 1 nanometer to 200 or 400 microns, for example from 1 to 200 microns.

In one embodiment, on and/or in the porous aluminium oxide layer there is a bioactive material. Suitable bioactive materials include bioactive glasses and ceramics, certain proteins and trace elements. Bioactive glasses and ceramics generally contain, apart from SiO₂, P₂O₅, calcium or magnesium, generally as oxide or fluoride, and another metal oxide such as Na₂O, K₂O, Al₂O₃ or B₂O₃. The molar ratio of Ca to P is preferably from 4 to 6, for example about 5 while the SiO₂ content is generally from 30 to 50 wt %, for example 40 to 60 wt %. The phosphorus and magnesium or calcium can alternatively be provided as the magnesium or calcium phosphate. Typical glasses include those derived from Na₂O—CaO—P₂O₅—SiO₂, such as Bioglass 45S5 (24.5 wt % Na₂O—25.5 wt % CaO—6 wt % P₂O₅—45%SiO₂) which is especially preferred. Suitable biomolecules which can be used include collagens and growth factors while suitable trace elements include magnesium, copper and zinc. Use of an aluminium alloy containing desired trace elements including phosphorous, zirconium, tantalum and niobium will result in the aluminium oxide layer containing these.

In another embodiment the aluminium oxide layer comprises phosphate, generally as aluminium phosphate. In general in this embodiment the layer will also contain pores but these can be closed to provide greater strength for the prosthesis. Indeed the strongest material will possess no pores—even if they were present at some stage during production. Of course in this embodiment the aluminium oxide layer can also possess pores containing bioactive material. Thus the porous layer formed will generally contain some aluminium phosphate; typically the layer will contain 2 to 20%, for example about 6 to 8% by weight phosphate ions. It is believed that the presence of this phosphate facilitates bone growth on the porous layer. Thus the bone cells tend to flatten out over the layer and start spreading pseudopodia which is important for proliferation. It is believed that the presence of phosphate assists this process.

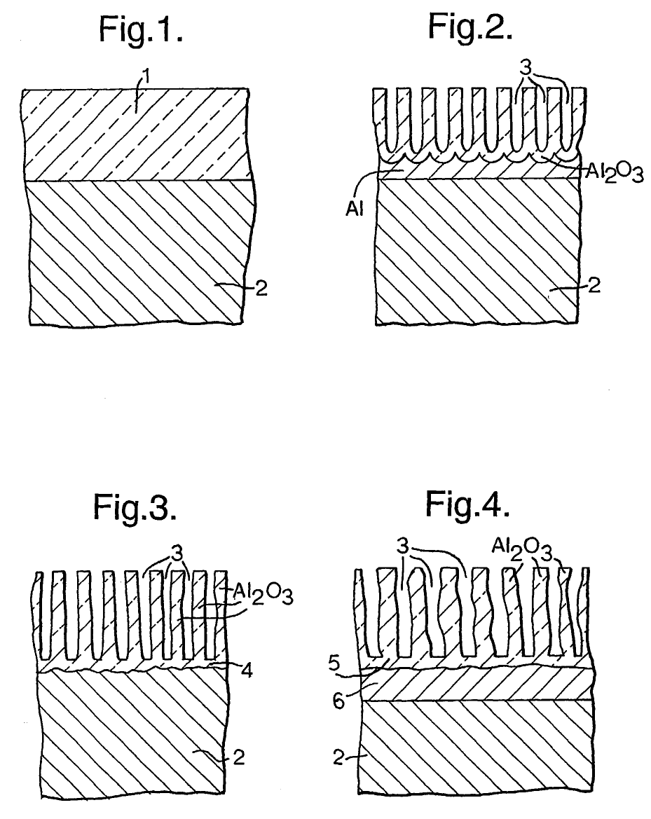

According to another aspect of the present invention, there is provided a process for preparing a prosthesis of the present invention which comprises coating at least a part of the metal surface of a prosthesis with aluminium or an alloy thereof and anodising the aluminium in an electrolyte which allows porous aluminium oxide to form and, if the electrolyte does not comprise phosphate, applying a bioactive material to the porous aluminium oxide. Of course bioactive material can be applied even if phosphate is present. The surface of the prosthesis, prior to aluminium deposition, can be textured with grooves, surface roughening or other features which aid fixation of the bone to the prosthesis. Coating the metal surface with aluminium can be carried out in any known manner including electroplating, electro-less plating, sputter coating, spray coating, DVD and vacuum evaporation, to provide an arrangement as shown in FIG. 1 of the accompanying drawings (1=aluminium coating; 2=implant). The precise nature of the method used is unimportant provided that a relatively fault-free layer is formed.

In an alternative embodiment the prosthesis is made of aluminium or an alloy of aluminium so that it can be anodised directly without the need for the initial coating step.

In order to convert the aluminium into a porous alumina layer, the aluminium is immersed in a bath of electrolyte that has some dissolving power for alumina and which is therefore capable of allowing a porous anodised layer to form. Typical electrolytes which can be used for this purpose include phosphoric acid, which is preferred, sulfuric acid, chromic acid and oxalic acid. In this arrangement, aluminium forms the anode and positive voltage is applied to it; the nature of the cathode is unimportant provided that it does not adversely affect the anode material.

In general using a phosphoric acid electrolyte, for example, the size of the pores which are formed will depend on the voltage used. Typically, a pore diameter of x nm with a pore spacing of 2.5x will result when a potential of x volts is applied to the metal. For a 0.16M oxalic acid electrolyte at 120V, 17 nm pores are produced with a 250 nm cell size and a maximum oxide thickness of about 1 mm can be produced. Typically, pores from 5 to 200 or 500 nm (diameter) will be produced and more generally from 50 nm to 0.3 microns, especially from 0.1 to 0.2 or 0.25 microns. The thickness of the anodised layer will depend on the length of time that the anodising process is carried out. It generally does not exceed 100 or 200 microns and is preferably 0.5 or 1 to 10 microns, typically 1 to 2 microns. In some instances it may be desirable for the anodised layer to be thicker than the preferred range so as to increase the potential interface between the bone and the implant coating. On the other hand it should be borne in mind that if the anodised layer is too thick the structure becomes too weak.

For some metals an electrical breakdown occurs if high voltages are applied to them in baths of electrolyte and consequently the anodising voltage should be reduced before the interface with the underlying metal is reached. In general, the maximum voltage is about 160, the minimum voltage is typically 5.

The anodising conditions are generally not otherwise critical. Direct current. is preferably used but alternating, pulsed or biased current may also be employed. The concentration of the electrolyte is typically 0.05 to 5M preferably 0.1 to 0.5 or 1M. In general higher voltages require more dilute electrolytes.

It has been found that it is important that the bath of electrolyte is strongly agitated during anodisation, for example by using. blow jets.

The anodisation is carried out until a significant thickness of the surface aluminium layer is converted to porous alumina to act as a substrate for bone growth. Anodisation may be stopped before all the aluminium layer has been consumed, as shown in FIG. 2 (3=pores); it will be noted that the pores are “coated” with alumina. Alternatively it may continue until the interface between the anodised material and unanodised material reaches the implant material beneath the original aluminium coating. Indeed if the underlying metal is anodisable as is the case with titanium and certain titanium rich alloys then anodisation will proceed into the underlying metal layer to produce a “barrier” anodised layer of this material. In this embodiment, all the metallic aluminium will have been consumed leaving only the biocompatible alumina, as shown in FIG. 3 (4=anodised implant material forming “barrier” layer). The thickness of the barrier layer will depend upon the particular composition of the metal used for the implant and the anodisation voltage applied. The thickness of this barrier layer does not greatly depend on the time of anodisation once a certain thickness has been achieved and so the anodising voltage can be applied for a time which is long enough to be sure that all the metallic aluminium has been consumed.

If the anodisation terminates within the aluminium layer i.e. not all the aluminium is consumed, then no gradual reduction of voltage is required. If the aluminium is anodised all the way through to the substrate metal and that metal withstands the full anodisation voltage then, again, no voltage reduction is required. However if it is desired to anodise through the complete aluminium layer and the substrate will only withstand a lower anodisation voltage then the voltage is desirably reduced before the substrate is reached. This can be carried out gradually either stepwise or smoothly as discussed in greater detail in EP-B-178831 which provides further information on the anodisation procedure.

If the underlying metal will not support a substantial electrolyte voltage but it is desired that all the surface aluminum layer is consumed then an intermediate layer, typically 1 micron thick, of a metal which can withstand such a voltage may be coated over the original metal layer before the aluminium layer. Typical intermediate layers are formed from titanium, tantalum, niobium and tungsten. This intermediate layer can also act as an impervious, protective interlayer for the core. Anodisation can then proceed right the way through the surface aluminum layer and into the intermediate layer to produce a barrier layer before anodisation is stopped, as illustrated in FIG. 4 (5=anodised intermediate layer material forming “barrier” layer; 6=intermediate layer). In all cases if desired an initial layer of, for example, electroless nickel can be applied to improve adhesion to the underlying substrate. Further an inert barrier layer of, for example, platinum or gold can then be applied.

If it is desired to increase the size of the pores of the surface alumina layer then this can be achieved, at the expense of the thickness of the pore walls, by etching the surface layer in a material that dissolves alumina. This can be achieved using a solution of a strong acid or alkali, typically sodium or potassium hydroxide at a concentration of, for example 0.01 to 1 normal.

Inorganic aluminium oxide membrane filters are commercially available and are well established substrates for cell culture. The porous aluminium oxide layer produced on the surface of the implants will have similar characteristics to such membranes. Experiments have shown that this surface is suitable for the formation of new bone. In a specific test a primary culture of human osteoblast (HOB) and osteoblast-like cells from the imrnortalised cell-line MG63 were seeded onto the substrates. The cells were incubated and viability tested (MTT, 3-(4,5-dimethylthiazole-2-yl)-2-5-diphenyltetrazolium bromide), after one, four and seven days.

Tests have shown that the cells do adhere to the substrate and that the number of cells adhering increases with time. Another observation was that the MG63 cells appeared to create a weaker bond to the substrate compared to the HOB-cells. The cells also showed positive for ALP (alkaline phosphatase), an enzyme-marker for osteoblast differentiation into bone making cells. Thus the first criterion for a substrate to be suitable for the formation of new bone is fulfilled.

It will be appreciated that the prosthesis will generally be made of metal i.e. the core is metal in order to provide sufficient strength. However it is also possible for the substrate to be made of other materials such as a plastics materials or a ceramic material where strength is less important. Suitable plastics materials include synthetic resins, fibre-reinforced composites, carbonaceous materials such as carbon fibres, for example resin-bonded carbon fibres as well as aramid resins. Specific examples include silicones, phenolic resins, melamine and acetate, styrene, carbonate, ethylene, propylene, acrylic, fluorocarbon, sulphone, amide, vinyl chloride and butadiene polymers including nylons, and ABS polymers. Such materials can be used where, for example, the prosthesis is a plate; an artificial tooth, typically made of a ceramic can also be provided where at least a part of the roots possesses the porous bioactive materials-containing coating. Naturally where the substrate is not of an appropriate metal it will need to be coated with such a metal so that the aluminium can be attached to it. This can generally be achieved electrolytically or by vacuum deposition. Generally it is desirable first to treat the surface of the plastics or other material so as to enhance the bond with the metal, for example by roughening or etching it. This can be achieved mechanically by, for example, dry abrasive blasting or wet abrasive tumbling, or chemically be etching with solvents, oxidising acids such as dichromate eg. sodium dichromate, or caustic solutions. Other methods include corona plasma etching which provides a “pock marked” surface and sputtering. In this last technique an adhesion promoter such as silicon monoxide, or hexonethane disiloxane can be used. The surface should desirably be continuous and not porous. It is envisaged that artificial cartilage and the like can be prepared in this way using a suitable plastics material.

The roughening step is typically followed by cleaning and sensitisation of the surface. Commercial sensitisation routes generally use a solution of stannous chloride in hydrochloric acid. However, other suitable sensitising solutions include gold chloride, palladium chloride, platinum, tin fluoroborate, silicon tetrachloride and titanium tetrachloride. It is important to remove all traces of the sensitising medium before the plating or metal evaporation step. A particular advantage of the method of this invention resides in the fact that relatively low temperatures are needed such that the use of plastics materials is possible.

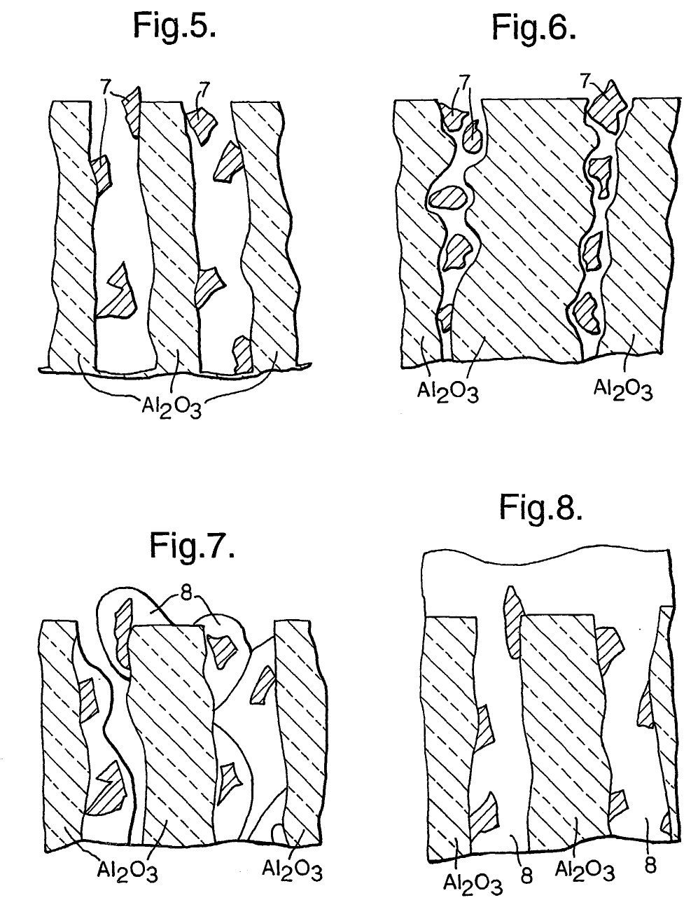

The ability of the porous aluminium oxide surface to bind with the bone is enhanced by the incorporation of bioactive material. As is known, such bioactive materials are excellent at promoting bone growth and the interface between them and the resulting bone can be as strong as bone itself. It is known that the stress caused in bone results in a Piezo electric effect which stimulates the bone to grow. A disadvantage of metal-based implants is that the metal present reduces this electric effect. However the incorporation of bioactive material in the porous layer enables the bone to grow onto the prosthesis. According to a further feature of the present invention bioactive materials are incorporated into the surface alumina layer. One method of achieving this is to form the bioactive material, for example bioactive glass, into particles of a size which can enter the pores of the surface alumina layer, as illustrated in FIG. 5 (7=bioactive material). A blend of particles can also be used. A plurality of different materials can also be used forming layers of such materials in the pores. Indeed in one embodiment one forms a layer of a highly bioactive material e.g. bioglass 45 which dissolves relatively quickly and would promote relatively rapid new bone growth and attachment and a layer of less bioactive material such as HA or other bioactive glass which dissolves more slowly and therefore is longer lasting. This latter material can therefore be placed at the base of the pores while the highly reactive material is at the top in contact with the bone thus giving bone growth a “kick start”. Indeed the use of an excess of such a highly reactive material such that it coats the surface as well as filling the pores can sometimes be tolerated if it dissolves comparatively quicldy. Again a mixture of such two differently reactive bioactive material could be used to fill the pores.

Other bioactive and bone promoting agents such as enzymes, hormones, proteins and other biomolecules can be incorporated using, for example, hyaluronic acid. The acid will trap the bioactive agents in the pores while allowing a slow release of bioactive material until bone has formed. Biomolecules can also be chemically attached to the pore walls using, for example, specifically tagged self assembling monolayers.

In the case of incorporation of particles, such particles typically have a size less than 0.1 microns, for example 1 to 200 nm, typically 2 to 50 nm, for example 2 to 10 nm, such as about 5 nm. They can be made by, for example, grinding in a ball mill, attrition milling and other techniques such as chemical synthesis which may be used for small sized particles. Thus it is possible to form a colloidal sol of, for example, bioactive glasses, silica or calcium phosphate e.g. hydroxyapatite. The sol particles can readily be made significantly smaller than the pores in the alumina layer. The particles can then be incorporated into the surface alumina layer using methods such as electrophoresis. In this case a voltage is applied to the central metal implant while it is immersed in a liquid containing the microscopic particles of bioactive material. The particles are attracted down the field lines until they reach the alumina surface where they are deposited. To improve pore filling, the liquid can agitated, for example using ultrasonic agitation, and/or the alumina surface can be wiped following the deposition. The nature of deposition is not important; alternative procedures include in situ precipitation. In general the porous alumina will possess a charge which will assist retention of the bioactive material.

If required, the bioactive material can be held more firmly in the pores by subsequent boiling in water. This will cause the pore walls to swell thereby applying pressure to the bioactive material. Alternative chemical methods which cause swelling can also be used. This is the same process as is used for producing, for example, anodised aluminium window frames in which case the solution contains a dye which is trapped in the pores as they are sealed by boiling or other chemical processes. In the present case the resulting reduction in pore diameter, thereby trapping the bioactive material, should be stopped before the pores are entirely sealed, as shown in FIG. 6.

If the electrolyte comprises phosphate then phosphate will be incorporated into the aluminium oxide layer thus making the use of bioactive material no longer essential. The concentration of phosphate in the layer may largely depend on the concentration in the electrolyte. However it should be noted that at the higher voltages used (generally for large size pores) there is a danger that too high a concentration will lead to electrical breakdown; this can be mitigated by cooling the electrolyte to, say, −5° C. It has been found that the concentration of phosphate in the layer sometimes varies such that the concentration is at its maximum at the walls of the pores and decreases as the distance from a pore wall increases.

In order to increase the strength of the phosphate-containing layer it is possible to close up the pores or even to cause them to collapse such that the layer is no longer porous. This can be achieved in known manner. Thus pore sealing can be achieved by heating in steam or water above about 70° C. Often boiling water can be used. Chemical methods for pore sealing include the use of nickel or cobalt acetate and nickel sulphate solutions as well as dichromate solution, typically 5-10% concentration Processes for the anodic oxidation of aluminium and aluminium alloy parts, DTD. 910C, HMSO, London 1951).

Placing the implant with the porous surface layer containing bioactive material in body fluids, either in vitro or in vivo may result in a surface of HA-like material (8) being seeded on the bioactive material as shown in FIG. 7. After continued exposure, the amount of HA-like material increases until, eventually, a continuous or nearly continuous layer of HA-like material is formed across the surface of the implant as shown in FIG. 8.

The prostheses can be used effectively to replace any bone which needs replacing or whenever a bone implant is needed. In addition, it can be used for dentures and also, according to a further aspect of the present invention, for artificial joints which involves a new style geometry. This new style geometry can be applied whenever the interface with the natural bone is sufficiently strong to support substantial tensile and shear stresses. A particular advantage of this aspect of the invention is that the stresses transferred to the bone more closely resemble those occurring in the natural joint system and so maintain the health of the underlying and adjacent bone. Although the invention is particularly directed at the growth of bone vesicles/cells it is also applicable to other types of tissue including cartilage and other forms of connective tissue.

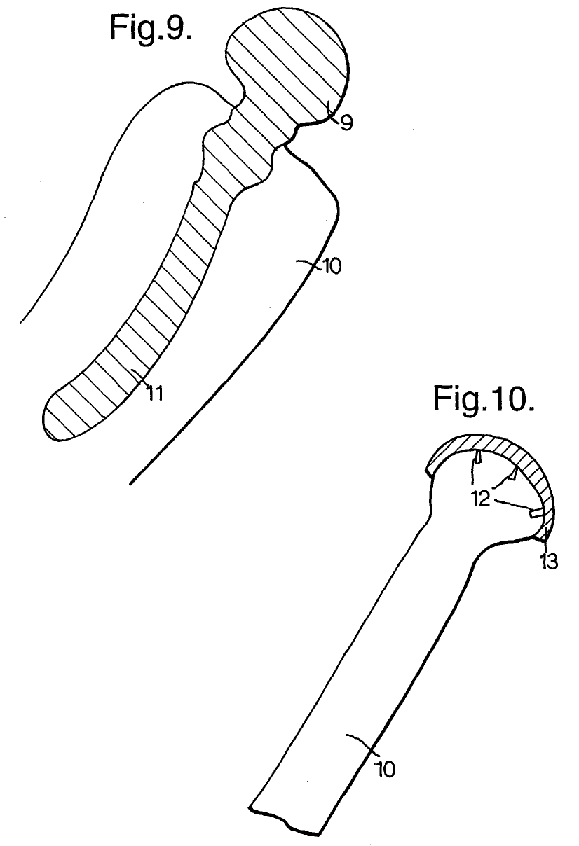

In accordance with the present invention, the long implant shaft (conventional metal implant), which can run many centimetres down a hole roughly in the centre of the bone and which is used to support a large artificial ball and socket type arrangement as shown in FIG. 9, is done away with (9=ball, 10=natural bone, 11=implant; socket part of joint not shown). Instead it is replaced by two co-operating thin roughly hemispherical caps that bond to the surface of the ball and socket parts of the natural joints, as shown in FIG. 10 (12=locating pins, 13=cap over natural ball joint; socket part ofjoint not shown. NB. Not to same scale as FIG. 9). The convex surface of one of the caps then slides inside the concave surface of the other to provide the joint motion. Surface coatings, for example of hyaluronic acid, can be applied to improve the wear properties of the joint. Ideally the surfaces of the artificial joints that move against each other during joint motion should comprise bioactive material that promotes the growth of natural cartilage. In this way natural cartilage will coat the rubbing surfaces so that any wear products are naturally absorbed into the body without causing damage to the surrounding bone.

Accordingly the present invention also provides a method of repairing a human or animal bone ball-and-socket joint which comprises optionally shaping the ball joint to receive a prosthesis in the form of a cap attaching the cap to the ball joint, and attaching a corresponding cup to the socket joint, optionally after shaping it, such that the concave surface of the cap and/or the convex surface of the cup is an aluminium oxide layer comprises phosphate and/or pores containing bioactive material thereby forming a prosthesis of the present invention. The present invention also provides a prosthesis for a ball and socket joint which comprises a cap which cooperates with a cup in which the cap and the cup are of metal, the concave surface of the cap and/or the convex surface of the cup bears an aluminium oxide layer which comprises phosphate and/or pores containing bioactive material optionally with a layer of aluminium or an alloy thereof between the metal of the cap and/or the cup and its porous aluminium oxide layer.

The bone surface can be prepared for application of the cap-like prosthetics by grinding to a surface radius of curvature the same as that of the prosthetic to be applied. This can be done on the “ball” side of the joint with a cup shaped grinder and with a ball shaped grinder on the “socket” part of the joint. In each case the radius of curvature of the prepared bone surface should closely match that of the prosthetic to be applied so that a strong interface with the natural bone is readily established. Small locating pins or screws are desirably used to hold the prostheses in place until the interface with the natural bone reaches adequate strength.

In one embodiment only one of the ball and socket bears the porous aluminium oxide layer. Preferably, though, both the ball and socket bear the layer to which can be adhered natural cartilage, as discussed above, so that the wear debris does not promote aseptic loosening.

If a long implant shaft is used, the present invention also provides an advantage. During the fitting process a hole is drilled down the centre of the femur, into which the implant is rammed. The fitting process creates a large amount of dead bone, and other tissue, which surrounds the implant after it is fitted. The presence of this dead tissue causes the body to attack the debris as being foreign and it is gradually reabsorbed, along with some of the surrounding healthy bone. As the implant becomes loosened fretting damage also takes its toll, (“aseptic loosening”). According to the present invention, any implant can be encouraged to grow bone into the debris field, and maintain this effect over several years. This would be very commercially attractive, as it could reduce the frequency of replacement operations. It is possible that a synergistic effect may be observed where the early production of a securely located implant leads to less fretting damage after several years, and a considerably extended lifetime.

The ability to form a bioactive layer on plastics based substrates could also allow artificial cartilage implants to be bonded to a bone substrate, for example in the knee. Thus an implant can be designed which has a bone promoting coating on one side and a cartilage cell promoting layer on the other. Other combinations of tissue are also possible.

1. A prosthesis at least a part of the surface of which is of metal, said metal being covered by a layer of aluminium oxide, optionally with a layer of aluminium or an alloy thereof between the metal and the aluminium oxide layer, and the aluminium oxide layer comprises either phosphate and/or pores containing bioactive material

2. A prosthesis according to claim 1 in which the metal is stainless steel, a Co—Cr alloy, titanium, a titanium alloy or aluminium.

3. A prosthesis according to claim 1 or 2 in which the aluminium oxide layer also comprises an oxide of one or more of magnesium, copper and zinc.

4. A prosthesis according to any one of claims 1 to 3 in which the aluminum oxide layer is one nanometer to 100 microns thick.

5. A prosthesis according to claim 4 in which the aluminium oxide layer is 1 to 100 microns thick.

6. A prosthesis according to any one of the preceding claims in which the aluminium oxide layer comprises pores having a diameter from 5 to 200 nanometer.

7. A prosthesis according to any one of the preceding claims in which the bioactive material is of glass or is a hydroxyapatite.

8. A prosthesis according to any one of claims 1 to 6 in which the aluminium oxide layer comprises phosphate and pores which are closed.

9. A prosthesis according to any one of the preceding claims in which the aluminium oxide layer comprises 2 to 20% by weight of phosphate.

10. A prosthesis according to any one of the preceding claims in which the core is made of metal.

11. A prosthesis according to any one of claims 1 to 9 in which the core is made of a plastics material-or a ceramic.

12. A prosthesis according to any one of the preceding claims in which there is no optional intermediate layer of aluminium or an alloy thereof.

13. A modification of a prosthesis as claimed in any one of claims 1 to 10 in which there is no optional aluminium layer but there is an anodised layer of the metal between the metal and the aluminium oxide layer.

14. A prosthesis according to any one of the preceding claims substantially as described with reference to any one of FIGS. 1 to 8 of the accompanying drawings.

15. A prosthesis for a ball and socket joint which comprises a cap which co-operates with a cup in which the cap and the cup are of metal, the concave surface of the cap and/or the convex surface of the cup bears an aluminium oxide layer which comprises phosphate and/or pores containing bioactive material, optionally with a layer of aluminium or an alloy thereof between the metal of the cap and/or the cup and its porous aluminium oxide layer.

16. A prosthesis according to claim 15 which has one or more the features of claims 2 to 12.

17. A prosthesis according to claim 15 substantially as described with reference to FIG. 10 of the accompanying drawings.

18. A prosthesis in the form of a cap or cup as defined in claim 15 or 16.

19. A process for preparing a prosthesis as claimed in any one of claims 1 to 14 which comprises coating at least a part of the metal surface of a prosthesis with aluminium or an alloy thereof, anodising the aluminium in an electrolyte which allows a porous anodised layer to form and applying, if the electrolyte does not contain phosphate, a bioactive material to said porous anodised layer.

20. A process according to claim 19 in which the anodisation is continued until the pores extend to the said metal.

21. A process according to claim 19 or 20 in which the metal is itself anodisable and anodisation is continued until the surface of said metal is anodised.

22. A modification of a process according to any one of claims 19 to 21 in which the said metal is aluminium or an alloy thereof and at least a part of the surface of the prosthesis is anodised in an electrolyte which allows a porous anodised layer to form.

23. A process according to any one of claims 19 to 23 in which the size of the pores is increased by etching the surface layer with a material that dissolves aluminium oxide.

24. A process according to any one of claims 19 to 23 in which particles of a bioactive material are applied to the porous aluminium oxide layer.

25. A process according to claim 24 in which the bioactive material is held more firmly in the pores by causing the pore walls to swell.

26. A process according to claim 19 or 22 substantially as hereinbefore described.

27. A prosthesis as defined in claim 1 whenever prepared by a process as claimed in any one of claims 19 to 26.

28. A method of repairing a human or animal bone ball-and-socket joint which comprises optionally shaping the ball joint to receive a prosthesis in the form of a cap, attaching the cap to the ball joint and attaching a corresponding cup to the socket joint, optionally after shaping it, such that the concave surface of the cap and/or the convex surface of the cup bears an aluminium oxide layer which comprises phosphate and/or pores containing bioactive material.
